(19) United States
(12) Patent Application Publication
(10) Pub. No.: US 2001/0051156 A1
(43) Pub. Date: Dec. 13, 2001

(54) Methods for inhibition of polyclonal B cell activation and immunoglobulin class switching to pathogenic autoantibodies by blocking CD1-mediated interactions

(76) Inventors: Defu Zeng, Palo Alto, CA (US); Samuel Strober, Portola Valley, CA (US)

Correspondence Address: PAMELA j. SHERWOOD, Bozicevic, Field and Francis LLP, Suite 200, 200 Middlefield Road, Menlo Park, CA, 94025, US

(21) Appl. No.: 09/844,544

(22) Filed: Apr. 27, 2001

Related U.S. Application Data
(60) Provisional application No. 60/200,285, filed on Apr. 28, 2000.

(51) Int. Cl. A61K 39/395

(52) U.S. Cl. 424/131.1

(57) ABSTRACT

Pathogenic polyclonal B cell activation and immunoglobulin class switching to pathogenic autoantibodies is inhibited by binding molecules that specifically interfere with CD1 antigen, but do not activate signaling (blocking agents), or by molecules that bind to the T cell antigen receptor on T cells that recognize CD1. When CD1 mediated signaling is thus blocked, the T cell response is diminished, resulting in reduced polyclonal B cell activation and reduced immunoglobulin class switching to pathogenic autoantibodies.

Systemic lupus erythematosus (SLE) is an autoimmune disease characterized by polyclonal B cell activation, which results in a variety of anti-protein and nonprotein autoantibodies (see Kotzin et al. (1996) Cell 85:303-306 for a review of the disease). These autoantibodies form immune complexes that deposit in multiple organ systems, causing tissue damage. SLE is a difficult disease to study, having a variable disease course characterized by exacerbations and remissions. For example, some patients may demonstrate predominantly skin rash and joint pain, show spontaneous remissions, and require little medication. The other end of the spectrum includes patients who demonstrate severe and progressive kidney involvement (glomerulonephritis) that requires therapy with high doses of steroids and cytotoxic drugs such as cyclophosphamide.

It appears that multiple factors contribute to the development of SLE. Several genetic loci may contribute to susceptibility, including the histocompatibility antigens HLA-DR2 and HLA-DR3. The polygenic nature of this genetic predisposition, as well as the contribution of environmental factors, is suggested by a moderate concordance rate for identical twins, of between 25 and 60%.

Many causes have been suggested for the origin of autoantibody production. Proposed mechanisms of T cell help for anti-dsDNA antibody secretion include T cell recognition of DNA-associated protein antigens such as histones and recognition of anti-DNA antibody-derived peptides in the context of class II MHC. The class of antibody may also play a factor. In the hereditary lupus of NZB/NZW mice, cationic IgG2a anti-double-stranded (ds) DNA antibodies are pathogenic. The transition of autoantibody secretion from IgM to IgG in these animals occurs at the age of about six months, and T cells play an important role in regulating the IgG production.

Disease manifestations result from recurrent vascular injury due to immune complex deposition, leukothrombosis, or thrombosis. Additionally, cytotoxic antibodies can mediate autoimmune hemolytic anemia and thrombocytopenia, while antibodies to specific cellular antigens can disrupt cellular function. An example of the latter, is the association between anti-neuronal antibodies and neuropsychiatric SLE.

Putting immunotherapy into practice is a highly desired goal in the treatment of human disease. The basis for immunotherapy is the manipulation of the immune response, particularly the responses of T cells. T cells possess complex and subtle systems for controlling their interactions, utilizing numerous receptors and soluble factors for the process.

A variety of biologic agents are under investigation as potential treatments for SLE. These products are designed to specifically interfere with immunologic processes, including T cell activation; T cell-B cell collaboration; production of anti-double-stranded DNA antibodies; deposition of anti-double-stranded DNA antibody complexes; complement activation, and immune complex deposition and cytokine activation and modulation. More aggressive interventions include gene therapy and stem cell transplantation. Immunomodulatory agents recently tested include thalidomide, AS101, 2′ chlordeoxyadenosine, mycophenolate mofetil, and bindarit. Additional pharmaceutical treatments include the mild androgen dehydroepiandrosterone, selective estrogen receptor modulators, and the prolactin inhibitor, bromocriptine.

The development of specific therapies that prevent the pathologic polyclonal activation of B cells, without compromising normal immune function, is of great interest for clinicians and patients. The present invention addresses this issue.

### Relevant Literature

Zeng et al. (1998) J. Exp. Med. 187:525-536 were able to induce a lupus-like disease in mice by the transfer of transgenic CD4+ or CD8+ T cells that recognize CD1, but could prevent the disease induction by coninjecting CD4-CD8- T cells from the marrow of the same donor animals.

Park et al. (1998) Semin. Immunol. 10:391-398 review the CD1 pathway of antigen presentation.

Vaughan et al. (1998) Nat. Biotech. 16:535-539 review the uses of human and humanized antibodies in the clinic.

A number of articles explore the structure and biology of CD1. Amano et al. (1998) J. Immunol. 161:1710-1717 describe the expression of CD1 on B cell subsets, finding both beta-2 microglobulin dependent and independent forms. Antigenic ligands of CD1 are discussed by Brossay et al. (1998) Immunol Rev 163:139-50. The role of CD1 is presenting lipid antigens is discussed by Hong et al. (1999) Immunol Rev 169:31-44; and Joyce et al. (1998) Science 279(5356):1541-4.

### SUMMARY OF THE INVENTION

Methods and compositions are provided for inhibiting pathogenic polyclonal B cell activation, which activation may include immunoglobulin class switching to pathogenic autoantibody isotypes. Of particular interest is the activation and class switching in B cells associated with the development of systemic lupus erythematosus. Binding molecules that specifically interact with CD1 antigen recognition, but do not activate signaling (blocking agents), are administered to a patient, and act to inhibit the function of T cells that recognize CD1. When CD1 mediated signaling is thus blocked, the T cell response is diminished, resulting in reduced polyclonal B cell activation and Ig class switching. Treatment with anti-CD1 monoclonal antibodies significantly delays the onset of proteinuria, reduces the levels of serum IgG and anti-dsDNA IgG and prolongs survival in a model system for SLE.

### BRIEF DESCRIPTION OF THE DRAWINGS

FIG. 1 shows a flow cytometric analysis of CD1 expression on splenic B cells. Spleen cells from 6-month-old C57BL/6 mice (A-C), 6-month-old NZB/NZW mice with proteinuria (D-F), 3-month-old C57BL/6 mice (G-I) and 3-month-old NZB/NZW mice (J-L) were stained with anti-B220-FITC or anti-IgM-FITC versus anti-CD1-Biotin (3C11 or 1B1) and counter-stained with streptavidin-PE. A subset of B220⁺CD1^{hi} or IgM⁺CD1^{hi}B cells is enclosed in the right box or upper-right box in each panel, and the percentage of CD1^{hi}B cells among live nucleated cells is shown for each box. The IGM⁻CD1^{hi}B cells are enclosed in the lower-right box in each panel. Each panel is representative of at least four replicate experiments.

FIG. 2 illustrates spontaneous secretion of IgM antibodies by CD1^{hi}B cells. Panel A shows gates for spleen cells from 6-month-old NZB/NZW mice without proteinuria after staining with anti-B220-FITC versus anti-CD1 (1B1)-PE and sorting into B220⁺CD1^{lo}, B220⁺CD1^{int} and B220⁺CD1^{hi} subsets. The percentage of cells amongst live nucleated cells is shown for each gate. Panels B and C show concentration of IgM and IgM anti-dsDNA antibodies, respectively, in culture supernatants of each subset (5×10⁵ cells/well) with or without syngeneic T cells (1.25×10⁵ cells/well). Data shows the Mean ±SE of six cultures from two experiments.

FIG. 3 shows the spontaneous secretion of IgM and IgG by IgM⁺ and B220⁺ B cells. Panels A and B show respectively the IgM and IgG production by sorted splenic B220⁺ and IgM⁺B cells (5×10⁵ cells/well) from 6-month old NZB/NZW mice with proteinuria (≧3+). Data shows the Mean±SE of six cultures from two experiments.

FIG. 4 illustrates the proliferation of T cells in response to stimulation by CD1 transfected A20 cells. Panels A and B show the expression of CD1 on A20 cells, a B cell lymphoma line, and CD1 transfected A20 cells (A20/CD1) by staining the cells with anti-B220 versus anti-CD1 mAbs. Panel C shows the proliferation of sorted splenic T (Thy1.2⁺) cells (1×10⁵/well) from 3-month old NZB/NZW mice co-cultured with the irradiated (5000 rads), graded numbers of A20 or A20/CD1 cells as measured with ³H-TdR incorporation. Each panel is representative of three replicate experiments.

FIG. 5 depicts the amelioration of lupus by in vivo anti-CD1 mAb treatment. Groups of 8-week old NZB/NZW mice were given 5 i.p. injections of anti-CD1 mAb or control rat IgG at a dose of 250 μg/mouse over a period of 30 days (days 0, 3, 5, 15 and 30). Thereafter, the mice were monitored with serum levels of IgG and anti-dsDNA IgG, and proteinuria and survival as shown in Panels A, B, C and D, respectively. There were 10 mice in each group. Arrows show time points of injections.

### DETAILED DESCRIPTION OF THE INVENTION

Pathogenic polyclonal B cell activation and/or class switching is inhibited by the administration of a blocking agent that interferes with T cell receptor binding of CD1, and prevents CD1 mediated immune cell activation. A disease of particular interest for the treatment methods of the present invention is systemic lupus erythematosus. Treatment with anti-CD1 monoclonal antibodies significantly delays the onset of proteinuria, reduces the levels of serum IgG and anti-dsDNA IgG and prolongs survival in a model system for SLE.

Definitions

It is to be understood that this invention is not limited to the particular methodology, protocols, cell lines, animal species or genera, constructs, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. Thus, for example, reference to “a construct” includes a plurality of such constructs and reference to “the cell” includes reference to one or more cells and equivalents thereof known to those skilled in the art, and so forth. All technical and scientific terms used herein have the same meaning as commonly understood to one of ordinary skill in the art to which this invention belongs unless clearly indicated otherwise.

Pathogenic polyclonal B cell activation and class switching: as used herein, this term refers to autoimmune diseases wherein the primary pathology results from polyclonal stimulation of B cells resulting in overproduction of antibodies, particularly autoantibodies, and more particularly autoantibodies of a pathogenic isotype. The inappropriate activation or class switching of B cells may result from one or more of: dysfunctional expression of B cell activating cytokines; loss of B cell tolerance; T cell recognition of autoantigens and immunogens; mimicry of self-antigens by exogenous antigens; and the like. Of particular interest are diseases that are associated with CD1 mediated antigen presentation, e.g. systemic lupus erythematosus.

Pathogenic class switching occurs in diseases such as lupus, when a particular class or subclass of Ig causes some or all of the manifestations of disease. During the course of an antibody mediated immune response, B cells are induced to undergo genetic rearrangements that results in “switching” of a variable region to different constant region sequence. IgM is the major class of antibody secreted into the blood in the early stages of a primary antibody response. Antibody class switching is induced by B-cell activators in the presence of cytokines. The identity of the heavy-chain class to which a B cell is switched is believed to be regulated by cytokines and B-cell activators at the level of transcription of unrearranged heavy chain constant genes.

In higher vertebrates there are five classes of antibodies, IgA, IgD, IgE, IgG, and IgM, each with its own class of heavy chain. In addition, there are a number of subclasses of IgG and IgA immunoglobulins; for example, there are four human IgG subclasses (IgG1, IgG2, IgG3, and IgG4). The various heavy chains impart a distinctive conformation to the hinge and tail regions of antibodies and give each class (and subclass) characteristic properties of its own.

It has been reported that autoantibodies in the MRL/Mp-lpr/lpr mouse model for lupus are relatively restricted to the IgG2a isotype (Eisenberg et al. (1987) J Immunol 139(3):728-33). F1 hybrids of New Zealand Black (NZB) and New Zealand White (NZW) mice are genetically predisposed to develop a lupus-like autoimmune disease characterized by IgG autoantibody production. Genetic mapping has shown that NZW genes act to class-switch the autoantibody response, an effect that contributes to disease in these animals (Vyse et al. (1996) J Immunol 157(6):2719-27).

Numerous reports have linked the IgG class of antibodies, and in particular the IgG3 subclass (the human counterpart of mouse IgG2a) with the development of lupus and related conditions in humans. As reviewed by Maddison (1999) Adv Exp Med Biol 455:141-5, antibodies in lupus patients belong primarily to the IgG1 and IgG3 subclasses of immunoglobulin, are high affinity, and occur in large amounts. Rubin et al. (1995) J Immunol 154(5):2483-93 found that chronic exposure to procainamide commonly elicited autoantibodies, but that on a population of patients, rapid switch to the IgG class occurred only in patients who went on to develop drug induced lupus. Amoura et al. (2000) Arthritis Rheum 43(1):76-84 found that antinucleosome antibodies of the IgG3 isotype were a marker of active SLE, in particular of lupus nephritis.

Disease manifestations may result from immune complex deposition, leukothrombosis; thrombosis; autoimmune hemolytic anemia and thrombocytopenia; disruption of cellular function by antibody binding and blocking; etc.

CD1: CD1 is a nonpolymorphic, class I MHC-like, non-MHC encoded molecule that may be found non-covalently associated with β₂-microglobulin (β₂m). In humans, five isoforms of CD1 have been identified (CD1a, b, c, d and e), and human B cells are known to express CD1c and CD1d. In mice, only the CD1d isoform has been identified. CD1 molecules have been demonstrated to be antigen-presenting molecules for glycolipid and hydrophobic peptides. A natural ligand of murine CD1d has been reported to be glycosylphosphatidylinositol (GPI).

The human and mouse isoforms of CD1 have been cloned and characterized as to their sequence. The sequence of human CD1a may be found in Genbank, accession number M28825. The sequence of CD1b may be found in PIR1 section of the Protein Sequence Database, release 64.00, Mar. 31, 2000, accession numbers B39957; B45801; and 179470 (Martin et al. (1987) Proc Natl Acad Sci USA 84(24):9189-93). The sequence of CD1c may be found in PIR1, accession numbers C45801; C39957; and 179472 (Aruffo and Seed (1989) J. Immunol. 143:1723-1730). Human CD1d may be found in Genbank, accession number J04142 (Balk et al. (1989) Proc. Natl. Acad. Sci. U.S.A. 86 (1), 252-256). Human CDle sequence may be found in Genbank, accession number X14975, X15110 (Calabi et al. (1989) Eur. J. Immunol. 19 (2), 285-292).

For immunization purposes the sequence of the CD1 polypeptide may be altered in various ways known in the art to generate targeted changes in sequence. The polypeptide will usually be substantially similar to the sequences provided herein, i.e. will differ by at least one amino acid, and may differ by at least two but not more than about ten amino acids. The sequence changes may be substitutions, insertions or deletions. Deletions may further include larger changes, such as deletions of a domain or exon, providing for active peptide fragments of the protein. Other modifications of interest include epitope tagging, e.g. with the FLAG system, HA, etc. Such alterations may be used to alter properties of the protein, by affecting the stability, etc.

For the purposes of the present invention, the CD1 blocking agent will bind to the CD1 protein present on antigen presenting cells of the patient being treated. That is, for human therapy the blocking agent will block human CD1; and the like. Because CD1 is not highly polymorphic a patient will generally express the wild-type protein as described above, although there may be exceptions where a patient expresses a variant form of the protein.

Agents may specifically block one or more of the human CD1 isoforms, particularly isoforms expressed on antigen presenting cells, e.g. CD1d. In an alternative embodiment, a cross-reactive blocking agent that recognizes common epitopes on all CD1 isoforms; or a cocktail of isoform specific agents; is used to generally block all isoforms present in the patient.

CD1 blocking agents: are molecules that interfere with the binding of CD1 by the T cell antigen receptor, for example by competitive or non-competitive binding to the extracellular domain of CD1, or to T cell antigen receptors that recognize CD1. Usually the binding affinity of the blocking agent will be at least about 100 μM. The blocking agent will be substantially unreactive with related molecules to CD1, e.g. Class I MHC antigens. Further, blocking agents do not activate CD1 signaling. Conveniently, this may be achieved by the use of monovalent or bivalent binding molecules.

In an alternative embodiment, the interaction between CD1 positive cells and CD1 specific T cells is blocked by elimination of the responsive T cell subset, for example by administering antibodies that react with such T cells, administration of targeted toxins, etc.

Blocking agents may be peptides, lipids, e.g. glycolipids, phospholipids, etc., either alone or in combination with a peptide, e.g. soluble CD1; small organic molecules, peptidomimetics, soluble T cell receptors, antibodies, or the like. Antibodies are a preferred blocking agent. Antibodies may be polyclonal or monoclonal; intact or truncated, e.g. F(ab′)₂, Fab, Fv; xenogeneic, allogeneic, syngeneic, or modified forms thereof, e.g. humanized, chimeric, etc.

In many cases, the blocking agent will be a polypeptide, e.g. antibody or fragment thereof; soluble CD1; etc., but other molecules that provide relatively high specificity and affinity may also be employed. Combinatorial libraries provide compounds other than oligopeptides that have the necessary binding characteristics. Generally, the binding affinity will be at least about 10⁻⁶, more usually about 10⁻⁸ M, i.e. binding affinities normally observed with specific monoclonal antibodies.

Binding Assays: Candidate blocking agents are screened for their ability to block CD1 mediated activation. Assays to determine affinity and specificity of binding are known in the art, including competitive and non-competitive assays. Assays of interest include ELISA, RIA, flow cytometry, etc. Binding assays may use purified or semi-purified CD1 protein or T cell antigen receptor protein, or alternatively may use cells that express CD1 or T cell antigen receptor; cells transfected with an expression construct for CD1 or T cell antigen receptor, etc. As an example of a binding assay, purified CD1 or T cell antigen receptor protein is bound to an insoluble support, e.g. microtiter plate, magnetic beads, etc. The candidate blocking agent is combined with the bound CD1, and the binding is determined by reference to a competitor molecule, e.g. soluble CD1; CD1 specific antibody, soluble T cell receptor, etc.

Generally, a soluble monovalent or bivalent binding molecule will be less likely to activate CD1 signaling than a comparable polyvalent molecule. A functional assay that detects B or T cell activation may be used for confirmation. For example, a population of T cells may be stimulated with irradiated allogeneic cells expressing CD1, in the presence or absence of the candidate blocking agent. An agent that blocks CD1 signaling will cause a decrease in the T cell activation, as measured by polyclonal B cell activation; lack of T cell proliferation and cell cycle progression, release of IL-2, etc.

A number of screening assays are available for blocking agents. The components of such assays will typically include CD1, e.g. as a purified protein, present on antigen presenting cells, etc. The assay mixture will also comprise a candidate blocking agent. Generally a plurality of assay mixtures are run in parallel with different agent concentrations to obtain a differential response to the various concentrations. Typically, one of these concentrations serves as a negative control, i.e. at zero concentration or below the level of detection.

Conveniently, in these assays one or more of the molecules will be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule which provides for detection, in accordance with known procedures.

Assays of interest are directed to agents that block the binding of CD1 to its counter-receptors, e.g. the T cell antigen receptor. The assay mixture will comprise at least a portion of the natural counter-receptor, or an oligopeptide that shares sufficient sequence similarity to provide specific binding, and the candidate blocking agent, e.g. antibody, glycolipid, glycolipid, soluble T cell receptor, soluble CD1, etc. The oligopeptide may be of any length amenable to the assay conditions and requirements, usually at least about 8 amino acids in length, and up to the full-length protein or fusion thereof. The CD1 may be bound to an insoluble substrate. The substrate may be made in a wide variety of materials and shapes e.g. microtiter plate, microbead, dipstick, resin particle, etc. The substrate is chosen to minimize background and maximize signal to noise ratio. Binding may be quantitated by a variety of methods known in the art. After an incubation period sufficient to allow the binding to reach equilibrium, the insoluble support is washed, and the remaining label quantitated. Agents that interfere with binding will decrease the detected label.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl, sulfhydryl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

A variety of other reagents may be included in the screening assay. These include reagents like salts, neutral proteins, e.g. albumin, detergents, etc which may be used to facilitate optimal protein-DNA binding and/or reduce non-specific or background interactions. Also reagents that otherwise improve the efficiency of the assay, such as protease inhibitors, nuclease inhibitors, anti-microbial agents, etc. may be used.

Antibodies: Suitable antibodies for use as blocking agents are obtained by immunizing a host animal with peptides comprising all or a portion of CD1 protein. Suitable host animals include mouse, rat sheep, goat, hamster, rabbit, etc. The origin of the protein immunogen may be mouse, human, rat, monkey etc. The host animal will generally be a different species than the immunogen, e.g. mouse CD1 used to immunize hamsters, human CD1 to immunize mice, etc. Peptides derived from such highly conserved regions may be used as immunogens to generate cross-specific antibodies.

The immunogen may comprise the complete protein, or fragments and derivatives thereof. Preferred immunogens comprise all or a part of the extracellular domain of human CD1, where these residues may contain the post-translation modifications, such as glycosylation, found on the native CD1. Immunogens comprising the extracellular domain are produced in a variety of ways known in the art, e.g. expression of cloned genes using conventional recombinant methods, isolation from T cells, sorted cell populations expressing high levels of CD1, etc.

Where expression of a recombinant or modified protein is desired, a vector encoding the desired portion of CD1 will be used. Generally, an expression vector will be designed so that the extracellular domain of the CD1 molecule is on the surface of a transfected cell, or alternatively, the extracellular domain is secreted from the cell. When the extracellular domain is to be secreted, the coding sequence for the extracellular domain will be fused, in frame, with sequences that permit secretion, including a signal peptide. Signal peptides may be exogenous or native.

When the CD1 is to be expressed on the surface of the cell, the coding sequence for the extracellular domain will be fused, in frame, with sequences encoding a peptide that anchors the extracellular domain into the membrane and a signal sequence. Such anchor sequences include the native CD1 transmembrane domain, or transmembrane domains from other cell surface proteins, e.g. CD4, CD8, sIg, etc. Mouse cells transfected with the human CD1 gene may be used to immunize mice and generate antibodies specific for the human CD1 protein.

Monoclonal antibodies are produced by conventional techniques. Generally, the spleen and/or lymph nodes of an immunized host animal provide a source of plasma cells. The plasma cells are immortalized by fusion with myeloma cells to produce hybridoma cells. Culture supernatant from individual hybridomas is screened using standard techniques to identify those producing antibodies with the desired specificity. Suitable animals for production of monoclonal antibodies to the human protein include mouse, rat, hamster, etc. To raise antibodies against the mouse protein, the animal will generally be a hamster, guinea pig, rabbit, etc. The antibody may be purified from the hybridoma cell supernatants or ascites fluid by conventional techniques, e.g. affinity chromatography using CD1 bound to an insoluble support, protein A sepharose, etc.

The antibody may be produced as a single chain, instead of the normal multimeric structure. Single chain antibodies are described in Jost et al. (1994) J.B.C. 269:26267-73, and others. DNA sequences encoding the variable region of the heavy chain and the variable region of the light chain are ligated to a spacer encoding at least about 4 amino acids of small neutral amino acids, including glycine and/or serine. The protein encoded by this fusion allows assembly of a functional variable region that retains the specificity and affinity of the original antibody.

Human and humanized antibodies: For in vivo use, particularly for injection into humans, it is desirable to decrease the antigenicity of the blocking agent. An immune response of a recipient against the blocking agent will potentially decrease the period of time that the therapy is effective. There are several methods that may be pursued to provide human or humanized antibodies, including production of human antibodies in transgenic animal hosts, modification of animal antibodies to “humanize”, or “resurface” the antibody; or selection of human antibody fragments in a phage display screening. A review of human and humanized antibodies may be found in Vaughan et al. (1998) Nat. Biotech. 16:535.

Human antibodies may be raised by immunizing mice that have been genetically altered in inactivate the endogenous mouse Ig, and by the introduction of unrearranged human Ig loci (Bruggemann (1997) Curr. Opin. Biotechnol. 8:455-458; Jacobovits (1995) Curr. Opin. Biotechnol. 6:561-566; Mendez et al. (1997) Nat. Gen. 15:146-156). An advantage of this system is that whole monoclonal antibodies are produced by the animals.

Phage display of human antibodies utilizes screening assays of peptide fragments on the surface of filamentous phage (McCafferty et al. (1990) Nature 348:552-554). Strategies for selection include selecting for high affinity and/or neutralization potential, for receptor agonists, etc. Selection procedures are extremely flexible. After the initial selection, methods may be used for directed evolution of affinity in order to improve neutralizing or binding properties (Hoogenboom et al. (1995) Trends in Biotechnology 15:62-70). Phage display may utilize single chain antibodies where both heavy and light domain are presented, Fab fragments where only the heavy chain variable region is present, or may utilize periplasmic association of non-covalently bound heavy and light variable domain. In order to extend the half-life of the protein in vivo, the protein may be modified by cloning into mammalian expression vectors; pegylation (Chapman et al. (1999) Nat. Biotech. 17:780-783); and the like.

Methods of humanizing antibodies are also known in the art. The antibody of interest may be engineered by recombinant DNA techniques to substitute the CH1, CH2, CH3, hinge domains, and/or the framework domain with the corresponding human sequence (see WO 92/02190; Roguska et al. (1994) P.N.A.S. 91:969-973; Jones et al. (1986) Nature 321:522-525; Padlan (1991) Mol. Immunol. 28:489-498).

The use of Ig cDNA for construction of chimeric immunoglobulin genes is known in the art (Liu et al. (1987) P.N.A.S. 84:3439 and (1987) J. Immunol. 139:3521). mRNA is isolated from a hybridoma or other cell producing the antibody and used to produce cDNA. The cDNA of interest may be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant regions genes may be found in Kabat et al (1991) Sequences of Proteins of Immunological Interest, N.I.H. publication no. 91-3242. Human C region genes are readily available from known clones. The choice of isotype will be guided by the desired effector functions, such as complement fixation, or activity in antibody-dependent cellular cytotoxicity. Preferred isotypes are IgG1, IgG3 and IgG4. Either of the human light chain constant regions, kappa or lambda, may be used. The chimeric, humanized antibody is then expressed by conventional methods.

Antibody fragments, such as Fv, F(ab′)₂ and Fab may be prepared by cleavage of the intact protein, e.g. by protease or chemical cleavage. Alternatively, a truncated gene is designed. For example, a chimeric gene encoding a portion of the F(ab′)₂ fragment would include DNA sequences encoding the CH1 domain and hinge region of the H chain, followed by a translational stop codon to yield the truncated molecule.

Consensus sequences of H and L J regions may be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

Expression vectors include plasmids, retroviruses, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody may be joined to any strong promoter, including retroviral LTRs, e.g. SV-40 early promoter, (Okayama et al. (1983) Mol. Cell. Bio. 3:280), Rous sarcoma virus LTR (Gorman et al. (1982) P.N.A.S. 79:6777), and moloney murine leukemia virus LTR (Grosschedl et al. (1985) Cell 41:885); native Ig promoters, etc.

Formulations: The subject CD1 blocking agents are prepared as formulations at an effective dose in pharmaceutically acceptable media, for example normal saline, vegetable oils, mineral oil, PBS, etc. Therapeutic preparations may include physiologically tolerable liquids, gel or solid carriers, diluents, adjuvants and excipients. Additives may include bactericidal agents, additives that maintain isotonicity, e.g. NaCl, mannitol; and chemical stability, e.g. buffers and preservatives. or the like. The CD1 blockers may be administered as a cocktail, or as a single agent. For parenteral administration, the blocking agent may be formulated as a solution, suspension, emulsion or lyophilized powder in association with a pharmaceutically acceptable parenteral vehicle. Liposomes or non-aqueous vehicles, such as fixed oils, may also be used. The formulation is sterilized by techniques as known in the art.

Co-formulations: The subject CD1 blocking agent may be formulated or administered in conjunction with other agents that act to decrease polyclonal B cell activation or otherwise relieve the symptoms of SLE. These agents include non-steroidal anti-inflammatory drugs (NSAIDs), e.g. acetylsalicylic acid; ibuprofen; naproxen; indomethacin; nabumetone; tolmetin; etc. Corticosteroids are used to reduce inflammation and suppress activity of the immune system. The most commonly prescribed drug of this type is Prednisone. Chloroquine (Aralen) or hydroxychloroquine (Plaquenil) may also be very useful in some individuals with lupus. They are most often prescribed for skin and joint symptoms of lupus. Azathioprine (Imuran) and cyclophosphamide (Cytoxan) suppress inflammation and tend to suppress the immune system. The side effects of these drugs include anemia, low white blood cell count, and increased risk of infection. Other agents, e.g. methotrexate and cyclosporin are used to control the symptoms of lupus. Both are immunomodulating drugs which have their own side effects. Anticoagulants are employed to prevent blood from clotting rapidly. They range from aspirin at very low dose which prevents platelets from sticking, to heparin/coumadin.

### METHODS OF USE

The term “treatment” or “treating” means any treatment of a disease in a mammal, including SLE in human, and animal models of lupus. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; and/or relieving the disease, that is, causing the regression of clinical symptoms by administration of a protective composition after their initial appearance.

It will be understood that in human medicine, it is not always possible to distinguish between “preventing” and “suppressing” since the ultimate inductive event or events may be unknown, latent, or the patient is not ascertained until well after the occurrence of the event or events. Therefore, it is common to use the term “prophylaxis” as distinct from “treatment” to encompass both “preventing” and “suppressing” as defined herein. The term “treatment,” as used herein, is meant to include “prophylaxis.”

The term “effective amount” means a dosage sufficient to provide treatment for the disease state being treated. This will vary depending on the patient, the disease and the treatment being effected. CD1 blocking agents are used for the treatment of SLE; and can be used in co-formulations, e.g. as asteroid sparing agent to facilitate use of lower prednisone doses.

In vivo activity for the treatment of SLE may be demonstrated by testing a CD1 blocking agent in one of several strains of inbred mice with inherited lupus-like disease, observing for the appearance of ANA production, pathogenic anti-ds DNA antibodies, immune complex glomerulonephritis, lymphadenopathy, and abnormal B and T cell function mimicking the human situation, in control and treated groups. Human clinical efficacy is demonstrated in clinical trials, employing methodology known to those skilled in the art.

Various methods for administration may be employed. The CD1 blocking agent formulation may be injected intravascularly, subcutaneously, peritoneally, etc. The dosage of the therapeutic formulation will vary widely, depending upon the nature of the disease, the frequency of administration, the manner of administration, the purpose of the administration, the clearance of the agent from the host, and the like. The dosage administered will vary depending on known factors, such as the pharmacodynamic characteristics of the particular agent, mode and route of administration, age, health and weight of the recipient, nature and extent of symptoms, concurrent treatments, frequency of treatment and effect desired. The dose may be administered as infrequently as weekly or biweekly, or fractionated into smaller doses and administered daily, semi-weekly, etc. to maintain an effective dosage level. Generally, a daily dosage of active ingredient can be about 0.1 to 100 mg/kg of body weight. Dosage forms suitable for internal administration generally contain from about 0.1 mg to 500 mgs of active ingredient per unit. The active ingredient may vary from 0.5 to 95% by weight based on the total weight of the composition.

The following examples are offered by way of illustration and not by way of limitation.

### EXPERIMENTAL

### Example 1

The role of CD1 in the development of lupus in NZB/NZW mice was examined. The results show that IgM⁺CD1^{hi}B cells from the NZB/NZW spleen spontaneously secreted IgM and IgM anti-dsDNA autoantibodies at levels five to 25 fold higher than CD1^{int/lo}B cells. CD1-reactive T cells were present in the spleen of NZB/NZW mice also. In vivo anti-CD1 mAb treatment reduced the peak levels of serum IgG and IgG anti-dsDNA antibodies, delayed the onset of proteinuria, and prolonged the survival period. These results demonstrate that CD1 is expressed on the precursors of IgM and IgG autoantibody-secreting B cells, and that the interaction between the CD1^{hi}B cells and CD1 reactive T cells play an important role in the pathogenesis of lupus.

Materials and Methods

Mice. C57BL/6 female mice were obtained from the Department of Comparative Medicine, Stanford University breeding facility. NZB/NZW female mice were purchased from The Jackson Laboratory (Bar Harbor, Me.).

Monoclonal Antibodies, Immunofluorescent Staining, Flow Cytometric Analysis and Sorting. Single cell suspensions of spleen cells, or bone marrow cells obtained from the femur and tibia were prepared and stained with mAbs as described previously (Zeng et al. (1997) Blood 90(1):453-63). Stainings were performed in the presence of anti-CD16/32 (2.4G2, Pharmingen, San Diego, Calif.) at saturation to block FcRγII/III receptors, and propidium iodide (Sigma Chemicals, St. Louis, Mo.) was added to staining reagents to exclude dead cells. Erythrocytes were excluded by light scatter gating. FACS® analysis and sorting were performed with a FACS® Vantage (Becton-Dickinson, Mountain View, Calif.), and data was analyzed using FlowJo software. The purity of sorted cells was >98%.

The following conjugated mAbs were used for staining: FITC- and PE-anti-B220 (RA3-6B2), FITC-anti-IgM (R6-60.2), PE-anti-CD1(1B1), biotinylated-anti-CD1 (1B1), PE-streptavidin and biotinylated-rat-IgM(R4-22) purchased from Pharmingen, San Diego. Biotinylated-anti-CD1 (3C11) was purified and conjugated as described previously (Zeng et al. (1998) supra.)

In vitro secretion of IgM and IgG. Sorted splenic T and/or B cells were incubated in 96-well flat-bottom plastic plates in complete RPMI medium with 10% fetal bovine serum for 1-5 days at 37° C. in 5% CO₂. At the end of the culture period, supernatants were harvested and the concentrations of IgM and IgG were measured with the ELISA, using affinity purified goat-anti-mouse heavy chain specific antibodies as described below.

In vitro activation of B cells by cross-linking CD1. Sorted B cells (B220⁺) were first incubated with biotinylated-anti-CD1 (3C11) or biotinylated-rat IgM (R4-22, Pharmingen, San Diego) for 30 minutes; washed once and incubated with streptavidin for 30 minutes. Cells were washed again three times and incubated in 96-well flat-bottom plastic plates for 5 days. Thereafter, supernatants were harvested and IgM and IgG levels were measured with the ELISA described below.

ELISA assay of total IgM and IgG, and anti-double-strained (ds) DNA IgM and IgG. Measurements of total IgM and IgG in culture supernatants or sera were performed using an ELISA assay with goat anti-mouse IgM plus IgG (H+L chain) antibodies (Southern Biotechnology Associates, Birmingham, Ala.) to capture mouse IgM and IgG, and alkaline phosphatase labeled affinity-purified goat antibodies specific for mouse Ig isotypes (Southern Biotechnology Associates, Inc.) for detection as described previously (Zeng et al. (1998) supra.)

IgM and IgG anti-dsDNA antibodies were captured using deproteinized calf thymus DNA as described previously (Singh et al. (1995) J Exp Med. 181(6):2017-27),and ELISA was performed as described above. Anti-dsDNA titers are expressed as units/ml, using a reference positive standard of pooled serum from 6-7 month old NZB/NZW mice. A 1:100 dilution of this standard serum was arbitrarily assigned a value of 100 u/ml.

In Vitro Proliferative Responses. Sorted splenic T cells (Thy1.2⁺B220⁻) were incubated (1×10⁵ cells/well) together with graded numbers (1-25×10³ cells/well) of irradiated (5,000 rads) stimulator cells. The latter were either the A20 B cell line derived from BALB/c mice or CD1-transfacted A20 B cells (Brossay et aL, supra.) Cells were cultured in 10% FCS complete RPMI medium in 96-well round-bottom plastic plates for 72 hours at 37° C. in 5% CO₂. ³H-thymidine (1 μCi/well) was added 24 hours before cells were harvested. ³H-thymidine (New England Nuclear, Boston, Mass.) incorporation was measured in a liquid scintillation counter (1205 Betaplate, Wallac, Turku, Finland). All assays were performed in triplicate wells with responder or stimulator cells alone or together.

In vivo treatment of anti-CD1 and control mAbs. Anti-CD1 mAb (rat IgG2b) was purified from the hybridoma 1B1 and anti-human lymphoma-idiotype mAb (rat IgG1) was purified from hybridoma R9A9. Hybridoma supernatants were purified using recombinant protein G agarose columns (Life Technologies, Grand island, N.Y.). Eluates were checked for purity by gel electrophoresis, and concentrations of mAb were determined by spectraphotometer. Eight-week old NZB/NZW mice were injected intraperitoneally five times with the anti-CD1 rat mAb or irrelevant rat IgG control at a dose of 250 μg/mouse over a period of 30 days (days 0, 3, 5, 15 and 30). Thereafter, the mice were monitored daily for survival , weekly for proteinuria and monthly for serum levels of IgM and IgG and anti-dsDNA antibodies. Proteinuria was measured on a 1 to 4+ scale using a colorimetric assay for albumin (Albustix, Miles, Inc., Elkhart, Ind.). Mice were considered to have proteinuria if at least two consecutive urine samples were >2+ (100 mg/dl). Serum levels of IgM and IgG and anti-dsDNA were measured with the ELISA as described above.

Results

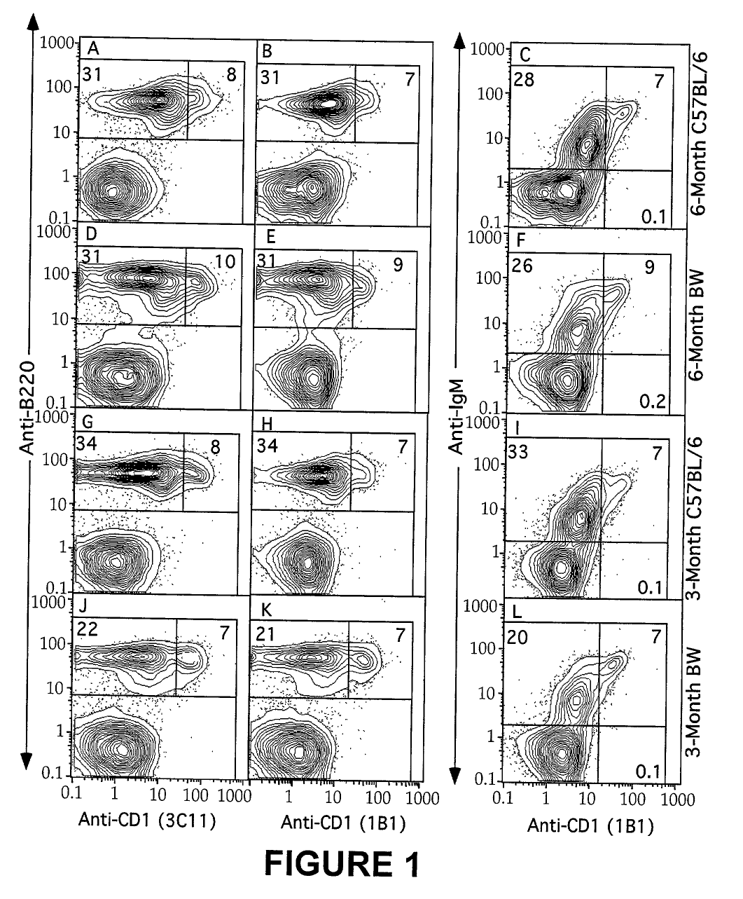

Splenic CD1 cells in NZB/NZW and C57BL/6 Mice The data provided herein compare the percentage of CD1^{hi}B cells in the spleen and bone marrow of lupus-prone NZB/NZW and non-autoimmune C57BL/6 mice at three and six months of age. As shown in FIGS. 1A and B, the spleen of C57BL/6 mice contained a subset of CD1^{hi}B cells, which accounted for about 7% of live nucleated spleen cells and about 20% of total spleen B cells, as judged by staining for CD1 receptors (using 3c11 and 1B1 mAbs) versus B220 receptors. The C57BL/6CD1^{hi}B cells were almost all IgM⁺ cells when anti-IgM mAb was used instead of anti-B-220 mAb (FIG. 1C). The percentage of CD1^{hi}B cells in the spleen of six-month old NZB/NZW mice with nephritis (proteinuria: ≧3+) was similar to that of the age- and sex-matched C57BL/6 mice. Almost all NZB/NZW CD1^{hi}B cells were IgM⁺ (FIG. 1D, E, F). The percentages of CD1^{hi}B cells in the spleen of C57BL/6 (G, H and I) and NZB/NZW (J, K and L) mice at three months were similar also.

The expression of CD1 on B220⁺ cells (B cells and their progenitors) in the bone marrow of C57BL/6 and NZB/NZW mice was studied. The CD1 expression level of the B220⁺ cells in the C57BL/6 bone marrow at age six months was more homogeneous than that in spleen, and almost all cells were CD1^{int/hi}. The percentage of B220⁺CD1^{int/hi} cells (23%) in the bone marrow of 6-month-old nephritic NZB/NZW mice was somewhat higher than that of age- and sex-matched C57BL/6 mice (16%). However, the percentage of IgM⁺CD1^{int/hi} B cells (11%) in the NZB/NZW bone marrow was five times higher than that in the C57BL/6 bone marrow (2%). The percentages of B220⁺CD1^{int/hi} and IgM⁺CD1^{int/hi} cells in the bone marrow of NZB/NZW mice were somewhat higher than that of C57BL/6 mice at the age of 3 months.

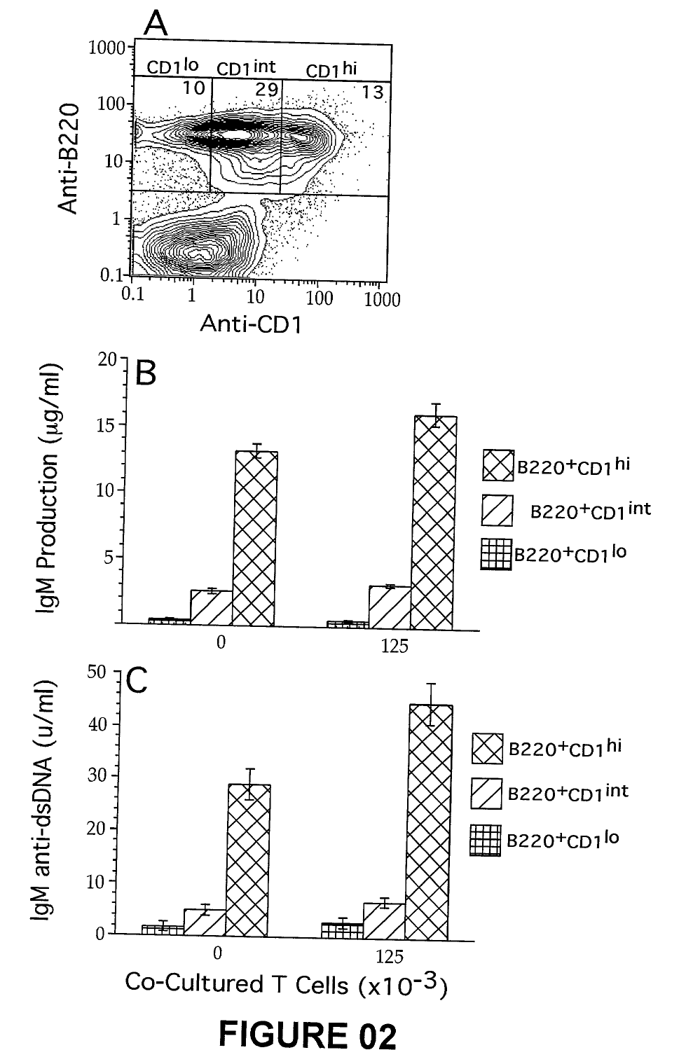

Splenic B cells from six month-old NZB/NZW mice spontaneously secrete large amounts of autoantibodies in vitro. In order to examine the role of CD1^{hi}B cells in secretion of autoantibodies, B cells from three- and six-month-old NZB/NZW mice without proteinuria or six-month-old mice with proteinuria were studied. The highest percentage of B220⁺B cells in the spleen was found in the six-month old mice without proteinuria. An example of the studies of splenic B cells from a group of the latter mice is shown in FIG. 2. Cells were sorted into B220⁺CD1^{hi}, B220⁺CD1^{int} and B220⁺CD1^{lo} populations using a non-activating anti-CD1 mAb (1B1) (FIG. 2 A). The cells of each population were cultured (5×10⁵ cells/well) in vitro with or without syngeneic co-cultured T cells (1.25×10⁵) for five days. Thereafter, the supernatants were assayed in duplicate for the concentrations of total IgM and IgG, and IgM and IgG anti-dsDNA antibodies. As shown in FIGS. 2B and C, CD1^{hi}B cells produced large amounts IgM (about 14 μg/ml) and IgM anti-dsDNA antibodies (about 30 u/ml) even without T cells in co-culture. This was five times higher than that secreted by CD1^{int}B cells (p<0.001, two-tail Student's t test) and 25 times higher than that secreted by CD1^{lo}B cells (p<0.001, two-tail Student's t test).

Add-back of syngeneic T cells significantly enhanced the IgM and IgM anti-dsDNA antibody secretion by the CD1^{hi}B cells (p<0.01, two-tail Student's t test). IgG secretion (<100 ng/ml) by the three subsets of B cells was too low to be compared. These results indicate that the IgM autoantibodies spontaneously secreted by splenic B cells of six-month-old NZB/NZW mice without proteinuria are predominantly derived from the CD1^{hi}B cell population. Studies using six-month-old NZB/NZW mice with proteinuria and three-month-old mice without proteinuria also showed that the CD1^{lo}B cells secreted little IgM autoantibody, and most was secreted by CD1^{int} and CD1^{hi}B cells. In addition, sorted C57BL/6 CD1^{hi}B cells secreted about 40 times less IgM (Mean±SE; 0.3±0.1 μg/ml) as compared to that secreted by NZB/NZW CD1^{hi}B cells (Mean±SE; 14±0.5 μg/ml).

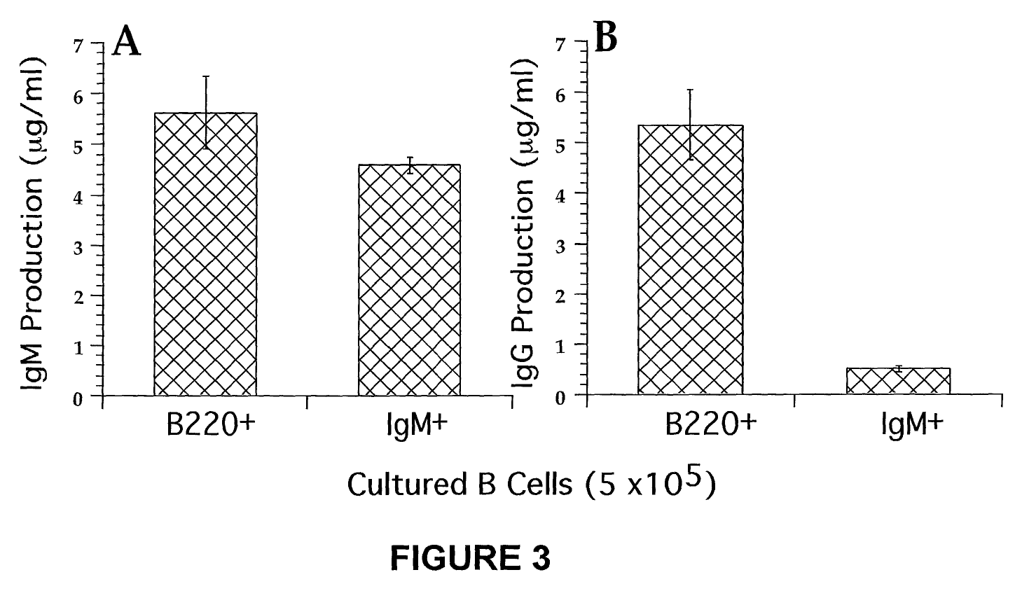

Glomerulonephritis and proteinuria are associated with spontaneous secretion of IgG anti-dsDNA antibody by splenic B cells. Splenic CD1^{hi}B cells from six-month-old NZB/NZW mice with proteinuria were almost all IgM⁺, and few, if any, were IgM⁻ (FIG. 1F). It was tested whether both IgM and IgG autoantibodies were secreted by IgM⁺B cells as compared to all B220⁺B cells from these mice. As shown in FIGS. 3A and B, while sorted B220⁺ cells produced large amounts (about 5.5 μg/ml) of both IgM and IgG, IgM⁺ cells produced large amounts (about 5.2 μg/ml) of IgM, but less than 0.5 μg/ml of IgG. Sorted residual B220⁺IgM⁻ cells were the source of IgG. There were insufficient CD1^{hi}IGM⁻ spleen cells to assay for spontaneous IgG secretion (FIG. 1F).

A kinetic study of in vitro IgM and IgG secretion was carried out and sorted B (B220⁺) cells from the spleens of six-month-old NZB/NZW mice with proteinuria were cultured and supernatants were harvested daily for 1 to 5 days with or without T cells. Sorted B cells (5×10⁵ cells/well) produced only about 0.2 μg/ml of IgM on day one, but the concentration of IgM increased gradually, and reached 3 μg/ml by day 5. Addition of T cells (1.25×10⁵) to cultures increased the secretion level by about two fold for each time point, but did not change the kinetic pattern. On the other hand, the kinetics of spontaneous secretion of IgG was different and no increase in the concentration was observed from day 1 through day 5 (about 1.7 μg/ml). Addition of T cells also increased the secretion level for each time point, but did not change the kinetics.

Previous studies showed that cross linking of CD1 receptors on BALB/c splenic B cells stimulated IgM secretion in vitro. In the present study, sorted B220⁺B cells from the spleens of three-month old NZB/NZW and C57BL/6 mice were incubated with a biotinylated IgM rat anti-CD1 mAb (3C11), then with streptavidin, and washed and cultured for five days. Supernatants were harvested thereafter. The sorted (B220⁺) B cells from NZB/NZW mice after exposure to an irrelevant rat IgM mAb produced about 3 μg/ml IgM. Cross-linking CD1 with the 3C11 mAb increased the IgM production by about two fold (p<0.01, two-tail Student's t test). On the other hand, the B cells from C57BL/6 mice spontaneously secreted little IgM (about 0.07 μg/ml) and cross-linking CD1 with 3c11 increased the IgM production by about 40 fold to 3 μg/ml (p<0.001, two-tail Student's t test).

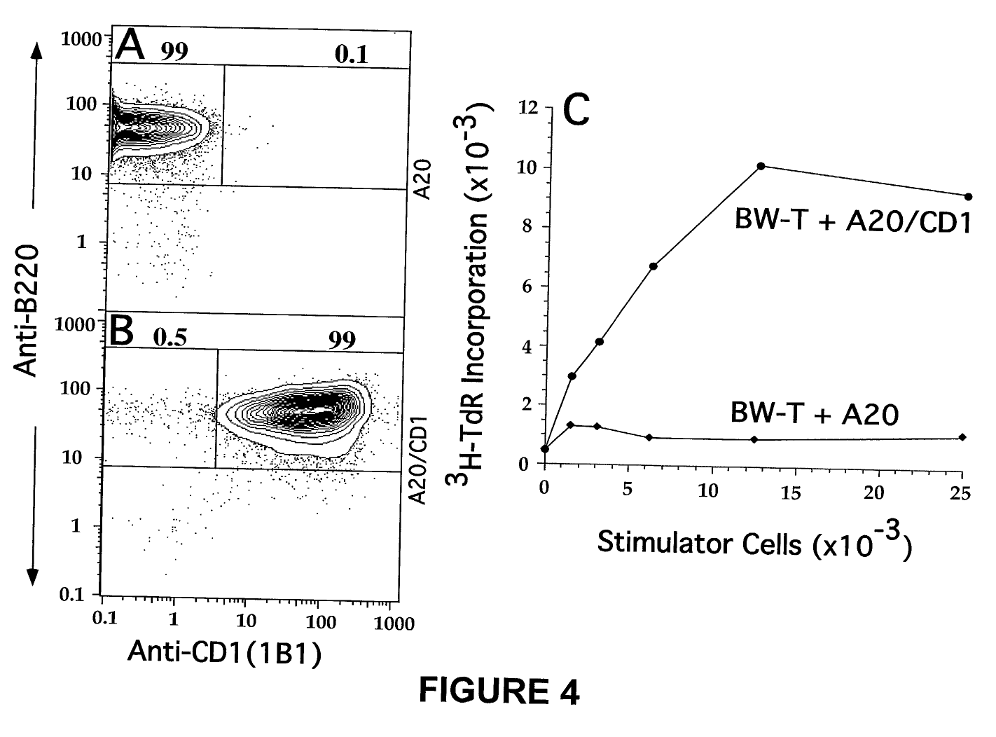

In order to determine whether anti-CD1 T cells are present in the spleen of three month old NZB/NZW mice, sorted splenic T (Thy-1⁺) cells were obtained from the latter mice and incubated with either the CD1-transfected A20 (A20/CD1) B cell line derived from BALB/c mice or the non-transfected A20 B cell line. As shown in FIG. 4, the sorted T cells proliferated vigorously in response to stimulation by A20/CD1 cells, but not to the A20 cells (FIG. 4C). A20 cells did not express CD1, but A20/CD1 cells expressed high levels of CD1 (FIGS. 4A and B). T cells from the spleens of C57BL/6 mice were not tested in this proliferation assay, because A20 cells (H-2^{d}) and C57BL/6 (H-2^{b}), T cells are MHC-mismatched, whereas NZB/NZW (H-2^{d/z}) T cells are not stimulated to proliferate by the shared H-2^{d} MHC of the BALB/c-derived cell line. In addition, the proliferation of the NZB/NZW T cells to A20/CD1 cells was about two-fold higher than that of BALB/c (H-2^{d}) T cells.

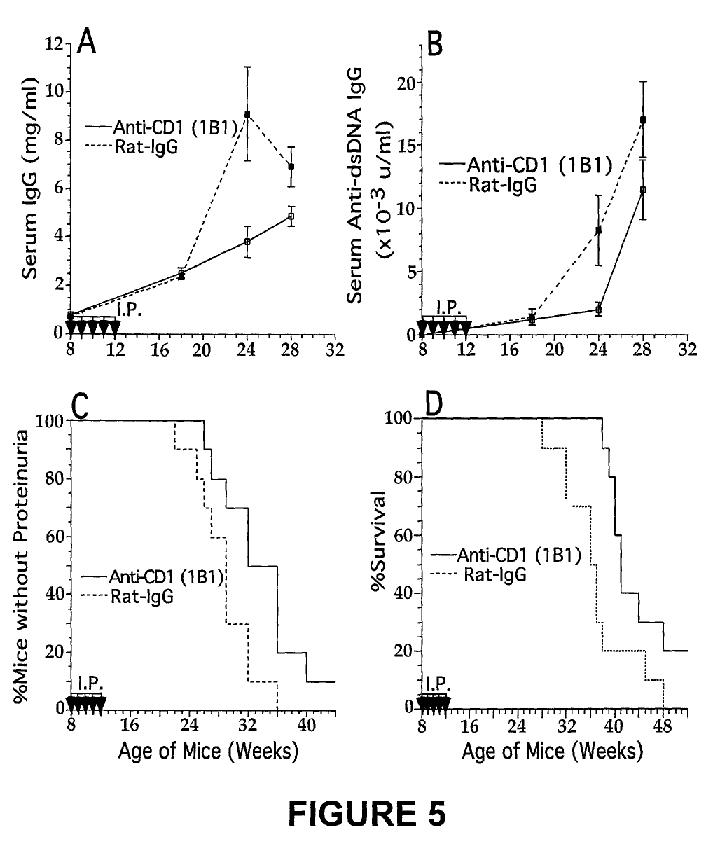

In vivo Anti-CD1 mAb treatment suppresses the development of lupus. Since anti-CD1 T cells were found in the spleen of NZB/NZW mice and CD1^{hi}B cells secreted IgM autoantibodies, it was possible that the T cells stimulated the secretion of IgM autoantibodies via CD1. Thereafter, the stimulated B cells could switch the autoantibody isotype from IgM to IgG associated with the development of disease. In order to interfere with B cell signaling via CD1 in a preliminary study, groups of eight-week old NZB/NZW mice were injected intraperitoneally five times over a 30-day period with 250 μg/mouse rat IgG anti-CD1 (1B1) mAb or control irrelevant rat IgG mAb (days 0, 3, 5, 15 and 30). Thereafter, the mice were monitored for levels of serum IgG and IgG anti-dsDNA, proteinuria and survival (FIG. 5). The anti-CD1 mAb was rat IgG_{2b}, a complement binding isotype, and the control mAb was rat IgG₁, a non-complement binding isotype. The control isotype was unlikely to worsen renal disease by the deposition of aggregate-complement complexes in the glomeruli.

As compared to control group, anti-CD1 treatment significantly reduced the peak levels of serum IgG and IgG anti-dsDNA autoantibodies (FIG. 5, A and B). At the age of 24 weeks, the mean serum IgG level (about 3000 μg/ml) of mice given anti-CD1 mAbs was three times lower than that (9000 μg/ml) of the mice given control rat IgG (p<0.01, two-tail Student's t test). The mean serum IgG anti-dsDNA antibody level at the same time point (about 1000 u/ml) was 8 times lower than that (8000 u/ml) of mice given control rat IgG (p<0.01, two-tail Student's t test). More than 4 weeks later, the differences in the mean levels of serum IgG and IgG anti-dsDNA antibodies of the two groups were reduced, but still remained significantly different (p<0.05, two-tail Student's t test). The serum levels of IgM and IgM anti-dsDNA antibody of the two groups increased during the observation period, however, the mean levels were not significantly different at all time points (p>0.4). Bleeding for serum samples was not done after 28 weeks of age due to the increasing morbidity and mortality after that time point, especially in the control group. The brief course of anti-CD1 mAb treatment also delayed the onset of proteinuria and prolonged the survival of the mice by four to eight weeks (FIG. 5, C and D). The differences in the onset of proteinuria (p<0.05, log rank test) and in survival (p<0.01, log rank test) were both significant. Thus, anti-CD1 mAb treatment delayed disease progression by at least the duration (four weeks) of the treatment period in this study.

In order to determine whether administration of anti-CD1 mAb depleted CD1^{hi}B cells, a single injection of 250 μg/mouse was given to three mice, and one week later, the spleen cells were assayed for the expression of CD1 on B cells. The staining pattern of CD1 versus B220 receptors and the absolute number of CD1^{hi}B cells was unchanged after the administration of the 1B1 mAb. Thus, the anti-CD1 mAb neither downregulated CD1 expression on B cells nor depleted CD1^{hi}B cells.

Considerable evidence suggests that the development of SLE has a strong genetic basis, which is a complex genetic trait with contributions from MHC and multiple non-MHC genes. Multiple genes in these disorders determine susceptibility, and no particular gene is necessary and sufficient for disease expression. H-2^{z} haplotype of the NZW parent line contributes to susceptibility, and the mechanism of this contribution is presumably via T cell recognition of pathogenic self-peptides presented by class II MHC molecules. T cells that recognize peptides derived from nucleosomes or anti-DNA antibodies augmented the secretion of pathogenic anti-DNA IgG antibodies. However, It is still not clear how conventional T cells recognizing peptides associated with MHC molecules provide help for B cells secreting those antibodies directed to a wide variety of non-protein antigens. One of the possible mechanisms is T cell recognition of the CD1 molecule associated with endogenous ligands on the surface of B cells. This interaction may induce polyclonal activation of B cells and augment the secretion of autoantibodies to non-protein antigens (i.e. nucleotides, phospholipids and phosphodiesters), since CD1 is a non-MHC encoded antigen-presenting molecule for non-protein antigens.

The above results show that the splenic B cells from the non-autoimmune C57BL/6 mice spontaneously produced little IgM. In contrast, the splenic B cells from the NZB/NZW mice without proteinuria spontaneouly produced large amounts of IgM, and those with proteinuria produced large amounts of IgM and IgG. T cells augmented the IgM and IgG autoantibody production. Furthermore, CD1^{hi}B cells in the spleen of NZB/NZW mice were found to be almost all IgM⁺, and they were the predominant source of in vitro spontaneous secretion of IgM autoantibodies as compared to CD1^{int} or CD1^{lo}B cells. However, those IgM⁺CD1^{hi}B cells were not the source of IgG autoantibodies, since IgM⁻B220⁺ cells mediated IgG secretion. The latter cells may have been derived from the former during the isotype switching to IgG with down regulation surface IgM.

CD1^{hi}B cells in the spleen of non-autoimmune C57BL/6 mice are predominantly marginal zone B cells, and they spontaneously secreted little IgM in vitro. Although the percentage of CD1^{hi}B cells in the spleen of NZB/NZW mice at age three and six months was found to be similar to the age and sex-matched C57BL/6 mice, the quality of the CD1^{hi}B cells from the NZB/NZW mice was markedly different from that in the C57BL/6 mice. The CD1^{hi}B cells in the spleen of NZB/NZW mice spontaneously secreted large amounts of IgM, a 40-fold increase as compared to that secreted by CD1^{hi}B cells from C57BL/6 mice. In addition, C57BL/6B cell secretion of IgM was increased more than 40 fold after incubation with anti-CD1 mAb, but NZB/NZW B cell secretion was only increased two fold. Although the CD1^{hi}B cells in the spleen of C57BL/6 mice have the surface phenotype of marginal zone B cells with little contribution from CD5⁺B (B-1) cells, splenic NZB/NZW CD1^{hi}B cells between ages three to six months have a markedly increased contribution from CD5⁺B (B-1) cells as compared to non-autoimmune mice, and these CD1^{hi}CD5⁺B cells spontaneously secrete IgM antibodies.

The kinetic study of in vitro autoantibody secretion by splenic B cells from the six-month old NZB/NZW mice with proteinuria showed that B cells produced large amounts of both IgM and IgG even without T cells in co-culture. But the kinetic patterns of IgM and IgG secretion were markedly different. The B cells (CD1^{hi}B) secreted little IgM on day 1, and the IgM secretion was increased gradually, and reached a 15-fold increase on day 5. On the other hand, IgG secretion by the B cells reached plateau on day 1 and no increase in concentration was observed from day 1 through day 5. Addition of syngeneic T cells increased the IgM and IgG production by two-fold at each time point, but did not change the kinetic patterns. These results indicate that in vitro secretion of IgM and IgG autoantibodies by splenic B cells from NZB/NZW mice are T cell independent, although T cells can augment the secretion. The kinetic pattern of IgM secretion suggests that the CD1^{hi}B cells can differentiate into IgM-secreting B cells in vitro even without T cell help. The engagement of CD40 on B cells and CD40L on T cells are crucial for B cell differentiation. The T cell independent differentiation of CD1^{hi}B cells may be explained by the observation that CD1^{hi}B cells from NZB/NZW spleen expressed high levels of both CD40 and CD40L. It is of interest that B cells from lupus patients were also found to express high levels of both CD40 and CD40L. It is possible that the upregulation of CD40L on autoimmune B cells is induced by the signals through CD1. The kinetic pattern of IgG secretion by the spleen B cells of NZB/NZW mice indicates that the isotype switch of autoantibody from IgM to IgG was completed in vivo and did not take place in vitro.

The presence of CD1-reactive T cells in the spleen of NZB/NZW mice was shown by experiments in which T cells from the NZB/NZW spleen proliferated vigorously to CD1-transfected B cells, but not to the parental non-ransfected B cells. Anti-CD1 mAb treatment was administered in vivo in order to interfere with the interaction between CD1-reactive T cells and CD1^{hi}B cells. The administration of the anti-CD1 mAb markedly reduced the peak levels of serum IgG and anti-dsDNA IgG. These results indicate that IgM anti-dsDNA antibody-secreting CD1^{hi}B cells may be the precursors of IgG anti-dsDNA antibody-secreting B cells, and the interaction of CD1 reactive T cells and the CD1^{hi}B cells via CD1 play an important role on the isotype switch of anti-dsDNA antibodies from IgM to IgG. This is consistent with a report that Th1-like CD1 reactive transgenic T cells induced lupus with high levels of serum anti-dsDNA IgG2a. It is of interest that CD1 reactive IL-4-procucing NK T cells that express the invariant Va14Ja281 TCRs are selectively reduced in NZB/NZW mice.

In conclusion, the above results demonstrate that the interaction between CD1^{hi}B cells and CD1 reactive T cells play an important role in the development of lupus, and that the progression of disease can be inhibited by the administration of blocking reagents that interfere with CD1 mediated signaling.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of treating pathogenic polyclonal B cell activation or class switching in a patient, the method comprising:
administering to said patient an effective dose of a CD1 blocking agent, wherein said blocking agent is characterized as interfering with T cell recognition of CD1 and is inhibitory of CD1 signaling;
wherein said dose is effective to treat the symptoms of said polyclonal B cell activation or class switching.

2. The method according to claim 1, wherein said pathologic polyclonal B cell activation or class switching results in systemic lupus erythematosus.

3. The method according to claim 2, wherein said CD1 blocking agent is a glycolipid or phospholipid.

4. The method according to claim 2, wherein said CD1 blocking agent is a polypeptide.

5. The method according to claim 4, wherein said polypeptide is an antibody or fragment thereof.

6. The method according to claim 5, wherein said antibody is a monoclonal antibody.

7. The method according to claim 6, wherein said monoclonal antibody is a human or humanized antibody.

8. The method according to claim 7, wherein said monoclonal antibody specifically binds to human CD1d.

9. The method according to claim 7, wherein said monoclonal antibody binds to multiple human CD1 isotypes.

10. The method according to claim 5, wherein said antibody comprises a cocktail of monoclonal antibodies that bind to multiple human CD1 isotypes.

11. The method of claim 4, wherein said polypeptide is soluble CD1 or a glycolipid bound to CD1.

12. The method according to claim 2, wherein said administration is by intravenous injection.

13. A method according to claim 2, further comprising administering to said patient a second therapeutic agent for the treatment of systemic lupus erythematosus.

14. The method according to claim 4, wherein said polypeptide is a soluble T cell antigen receptor.
